(19) United States
(12) Patent Application Publication
(10) Pub. No.: US 2018/0298441 A1
(43) Pub. Date: Oct. 18, 2018

(54) APPARATUSES AND METHODS FOR ASSESSING TARGET SEQUENCE NUMBERS

(71) Applicant: BioCeryx Inc., Menlo Park, CA (US)

(72) Inventors: Kirk Bradley, Menlo Park, CA (US); Robert Balog, Sunnyvale, CA (US)

(21) Appl. No.: 15/952,962

(22) Filed: Apr. 13, 2018

Related U.S. Application Data
(63) Continuation of application No. PCT/US2017/024098, filed on Mar. 24, 2017.
(60) Provisional application No. 62/313,454, filed on Mar. 25, 2016.
(60) Provisional application No. 62/345,586, filed on Jun. 3, 2016.

(51) Int. Cl. C12Q 1/6874; C12Q 1/6811; C12Q 1/682; C12Q 1/6827; C12Q 1/6806; C12Q 1/686; G06F 19/22; C12N 15/10

(52) U.S. Cl. CPC C12Q 1/6874; C12Q 1/6811; C12Q 1/682; C12Q 1/6827; C12Q 1/6806; C40B 30/04; G06F 19/22; C12N 15/1065; C12Q 2600/16; C12Q 2600/166; C12Q 2600/106; C12Q 1/686

(57) ABSTRACT

Embodiments in accordance with the present disclosure are directed to assessing for the presence of different target sequences in a sample. Embodiments include providing a binary result of the presence or absence of target sequences that is indicative of a disease or other physiological condition. An example method includes exposing a sample to a plurality of probes, the plurality of probes including a plurality of complimentary sequences that bind to a plurality of target sequences in the sample, and a plurality of different tag sequences for each of the plurality of target sequences in the sample. At least a portion of the target sequences bound to the probes are caused to bind to the different locations on the substrate. And, the method includes, by using scanning circuitry and information indicative of the different locations and associated tag sequences, assessing the number of the target sequences in the sample.

### OVERVIEW

Various embodiments in accordance with the present disclosure are directed to assessing for the presence and concentration of a plurality of different target sequences in a sample. In specific embodiments, a digital (microarray) technique is used to provide a binary result of the presence, absence, and/or relative or absolute copies or concentrations of one or more target sequences that is indicative of a disease or other physiological condition.

It can be advantageous for diagnosis of diseases or physiological conditions, as well as other analysis purposes, to detect, study, characterize and quantify nucleic acids in a biological sample. In accordance with various embodiments, a digital microarray process provides digital results (e.g., binary, such as “yes” or “no”) of the presence or absence of specific tag sequences that are combined to diagnose multiple diseases or physiological disorders from a sample that is automated, precise, and which can sense low concentrations of the target in the sample. With a traditional polymerase chain reaction (PCR) technique, such as with a thermal cycler, the detection of nucleic acids is performed at the end-point of the PCR reaction, is time consuming and non-automated, and yields results that are characterized by poor precision and low sensitivity. Other techniques, such as real-time PCR (qPCR), digital PCR and sequencing, are not ideal for multiplexing nucleic acid sequences, analyze one genomic target (with each droplet) and/or are time consuming and expensive to perform. PCR techniques are generally optimal when the number of sequences to be analyzed is less than 10 with time-consuming manual oversight, and sequencing (automated) techniques commonly involve analysis of a large number of sequences is large such as in excess of 100,000.

Microarrays provide another technique to study nucleic acids. Microarray readouts depend on measuring the fluorescent strength of a fluorescent signal emanating from a specific spot in the microarray. As an example microarray in this context, a microarray includes a collection of microscopic nucleic acid sequence spots (e.g., sequences) attached to a solid surface, such as a substrate or a surface of a substrate.

The digital (microarray) technique in accordance with aspects of the present disclosure can include a plurality of complementary tag sequences at different locations (e.g., unique locations) on the substrate that bind to hybridized genomic target sequences. Each tag sequence is measured using processing circuitry and scanning circuitry (e.g., microarray scanning circuitry). That tag measurement is then reduced to a binary value. Those binary values are then tallied (counted) for all of the tags associated with each target to generate a target count metric then is directly related to the initial concentration of the input sample. For example, a plurality of unique locations of the substrate (e.g., digital microarray) contain complementary tag sequences to tag sequences associated with a particular target. At the end of the PCR process, each unique location is analyzed to determine if the tag sequence is present or not (e.g., using florescent labels). In response to determining the tag sequence is present at a particular location, a bucket count indicative of the initial concentration of the target in the sample is increased by one. The final bucket count for each target quantifies the initial target concentration of the target in the sample.

One principle behind detection of the targets located on the substrate (e.g., microarray) is the hybridization between two sequences. Specifically, various embodiments include a substrate (e.g., digital microarray) with a plurality of complementary tag sequences on the surface of the substrate that bind to respective tag sequences of the probes. The complementary sequences specifically pair with each other by forming hydrogen bonds between complementary nucleotide base pairs. A high number of complementary base pairs in a genomic sequence means tighter non-covalent bonding between the two strands. After washing off non-specific bonding sequences, strongly paired strands remain hybridized. Fluorescently labeled tag sequences of the probes that bind to a complementary tag sequence generate a signal that depends on the hybridization conditions (such as temperature), and washing after hybridization. Total strength of the signal, from a spot (feature), depends upon the amount of target binding to the probes and the complementary tag sequence present on that spot. The relative quantitation in which the intensity of a feature is compared to the intensity of the same feature under a different condition, and the identity of the feature is known by its position (e.g., the property of complementary genomic sequences to specifically pair with each other by forming hydrogen bonds between complementary nucleotide base pairs).

The digital (microarray) technique can be applied to diagnostics that involve determining copy number variations between normal and diseased states. A variety of disease states and/or physiological conditions result in copy number variations in different nucleic acid biomarkers as compared to a normal state (e.g., a person that does not have the disease). While not limiting, examples of nucleic acid copy numbers variations can be found in multiple copies of entire chromosomes, multiple copies of specific genes within a chromosome, differential transcription of protein coding sequences (e.g., mRNA), and non-coding sequences (e.g., microRNA). Further, various embodiments includes the analysis of circular RNAs, and small non coding RNA to detect nucleic acid using the digital microarray technology (using the discovered nucleic acid biomarker classes).

In specific examples of a digital process, an input sample is provided with a plurality of genomic target sequences. The sample is exposed to a plurality of probes, such as by adding a plurality of probes to the sample. A target includes or refers to a nucleic acid sequence to be analyzed. Each probe includes the complementary sequence to the target sequence (and that can bind thereto) and a tag sequence whose complement is located in a particular location on a substrate (e.g., a unique or discrete microarray location). The plurality of probes include a plurality complimentary sequences that bind to the plurality of target sequences and a plurality of different tag sequences for each of the plurality of probes directed to one of the plurality of target sequences in the sample, with the different tag sequences binding to different locations on the substrate. For example, the plurality of probes for a given target include a plurality of copies of the complimentary sequence that binds to the given target sequence and a plurality different tag sequences each configured to bind to a different location on the substrate, such as an unique microarray location. In specific examples (as further illustrated herein by FIG. 3), the plurality of probes for a given target can include a set of M-probes with M-different tag sequences and the substrate that includes M-different complementary tag sequences. For example (as further illustrated herein by FIG. 4), the plurality of probes used to assess N target sequences can include N-sets of probes (and with the size of each probe set per target sequence being the same and/or different). The target sequences present in the sample bind to respective probes that have complementary sequences to the target, sometimes referred to as “hybridization.” After hybridizing to the probes, the number of bound targets in the sample is increased via an amplification process. For example, a PCR process in performed that amplifies a single or few copies of the amplicons (e.g., target sequences bound to a probe) across several orders of magnitude.

During hybridization, at least a portion of the amplified probe tag sequences (e.g., the probes bound to the target sequences) are caused to bind to their complementary tag sequence locations on the substrate, such as by the respective tag sequences of the probes (that are bound to a target sequence) binding to the complementary tag sequences located on the substrate. Sequences or other material in the sample that do not bind to the substrate or that do not bind to a probe can be removed. The number of each of the target sequences (e.g., a concentration or relative concentration) in the sample can be assessed using scanning circuitry and based on the information indicative of the different locations and associated tag sequences and/or target sequences. The assessment includes a binary assessment (i.e., presence or absence) of each tag sequence bound to the substrate, which are assessed by thresholding the intensity value returned by the scanning circuitry and indicative of the fluorescent signal of the hybridized tag sequence in the probe. For example, using information indicative of the different (e.g., unique) locations of the substrate and associated tag sequences, the number of the target sequences in the sample can be assessed by counting a number of tag sequences bound to the substrate that are associated with the target and based on captured fluorescence signals. The final assessment of each target can be the sum of all copies of the present tag sequences (known to be) associated with the target.

Various specific methods embodiments include analyzing approximately 10-10,000 molecules. In some specific embodiments, a concentration or relative concentration of a plurality of target sequences are determined that includes relatively small concentrations and/or small concentration differences between one another. For example, a concentration of at least one of the target sequences is determined based on a count (e.g., digital result) of number of (copies) and/or a count of tag sequences associated with the target sequence bound to the substrate using processing circuitry, which is indicative of copies of the target sequence present at different locations of the substrate. A digital result and/or output is provided for each of the plurality of different locations by capturing signal intensities at each location and providing a digital output (e.g., yes or no, 1 or 0) indicative of a present tag sequence or no tag sequence based on the same. The number of target sequence (e.g., copies of target sequences bound to probes which are bound to complementary tag sequences on the substrate) present on the substrate can be summed to provide the concentration or relative concentration of the target in the sample. The digital results reduce the time for detection and increase the precision and sensitivity to concentrations of targets, as compared to other techniques. For example, the digital results and/or concentrations determined can be used to detect amplification differences between amplicons and/or to determining when to stop the PCR reaction. By contrast, traditional microarray hybridization techniques are difficult to use to detect small changes in concentration as they generally rely on teasing out small concentration changes using relative probe intensity values for a sample containing a number of different target sequences or molecules. Further, when PCR is employed, small differences in concentration are often obscured by large differences in PCR efficiency between amplicons. The large differences in efficiency are inherent in the PCR process.

The above-described digital process can be implemented using one or more apparatuses. An apparatus can include processing circuitry, scanning circuitry, and optionally, a substrate and plurality of probes. As previously described, the substrate has a plurality of complementary tag sequence at a plurality of different locations. The complementary tag sequences can bind to different tag sequences of the plurality of probes. The probes include a set of probes for each target sequence (suspected to be or being tested for) in the sample. The scanning circuitry scans the substrate and, therefrom, capture signals indicative of tag sequences bound to the substrate. For example, the scanning circuitry captures fluorescent signal intensities of tag sequences bound to the substrate (e.g., a surface of the microarray). The processing circuitry assesses the number of each of the target sequences in the sample based on the captured signals and information indicative of the different locations and associated tag sequences and/or target sequences. The processing circuitry can use the captured fluorescent signal intensities to provide the digital output, as previously described. The apparatus can additionally include a microfluidic card with a plurality of chambers that are in fluidic connection and that are used to perform the hybridization of the probes to the targets in the sample, amplification, and hybridization of the amplicons to the substrate (e.g., a microarray), such as the rapid assay apparatus illustrated by FIGS. 8A-8C, illustrated on page 2 of the underlying Provisional Application (Ser. No. 62/313,454), entitled “Rapid Assay Process Development”, filed on Mar. 25, 2016, and illustrated on page 2 of the attached appendix of the underlying Provisional Application (Ser. No. 62/345,586), entitled “Digital Microassay”, filed on Jun. 3, 2016, each of which are which are fully incorporated herein by reference. In other embodiments, one or more additional apparatuses as used to perform the hybridization and amplification processes, such as various thermal cyclers.

The processing circuitry, in specific embodiments, provides a digital output using the captured fluorescent signals. The digital output includes or refers to a count for each of the plurality of different locations of the substrate. As described above, a concentration or relative concentration (e.g., copy number) for one or more of the target sequences can be provided using the digital outputs. For example, the processing circuitry determines a concentration of one or more of the target sequences in the sample based on a count (e.g., the digital output) of the number of each tag sequence associated with a respective target sequence bound to the substrate above a threshold intensity, and which is indicative of the number of copies of the target sequence present at the different locations of the substrate. The concentration can be determined by generating or identifying a target count score, referred to above as the “bucket count”, for the target sequences. To determine a target count score, the processing circuitry determines whether or not a tag sequence associated with the target sequence is present at each of the plurality of different locations of the substrate using the signal intensities captured by the scanning circuitry. The number of copies present on the substrate (e.g., a digital output indicative of “yes”) is summed by increasing the target count score by one responsive to determining a copy is present at the particular location (and not increasing by one in response to a copy not being present).

The target count scores can be used to diagnose an organism. For example, the sample obtained from the organism is used to provide the digital outputs and target count scores for a plurality of target sequences. The target count scores are compared to thresholds that are indicative of expected results for an organism that does not (or does) have a disease or other physiological disorder associated with the target sequences.

The above discussion is not intended to describe each embodiment or every implementation of the present disclosure. The figures and detailed description that follow also exemplify various embodiments.

### BRIEF DESCRIPTION OF THE FIGURES

Various example embodiments may be more completely understood in consideration of the following detailed description in connection with the accompanying drawings in the Appendix, which form part of this patent document.

FIG. 1A illustrates an unreacted molecular inversion probe in accordance with various embodiments of the present disclosure;

FIG. 1B illustrates a molecular inversion probe that is circularized and bound to a target sequence in accordance with various embodiments of the present disclosure;

FIG. 2A illustrates an example use of molecular inversion probes in a microarray process to identify different genomic targets in accordance with various embodiments of the present disclosure;

FIG. 2B illustrates a relationship between the concentration of a generic DNA molecule and PCR cycles in accordance with various embodiments of the present disclosure;

FIG. 3 illustrates an example use of molecular inversion probes to determine concentration of a single target in accordance with various embodiments of the present disclosure;

FIG. 4 illustrates the use of molecular inversion probes to determine the concentration of a plurality of targets in accordance with various embodiments of the present disclosure;

FIG. 5 illustrates an example experimental embodiment of a target capture with a plurality of probes in accordance with various embodiments of the present disclosure;

FIG. 6 illustrates an example process for providing a digital result for a disease or condition using a digital microarray, in accordance with various embodiments of the present disclosure;

FIG. 7 illustrates an example apparatus used for assessing target sequence numbers, in accordance with various embodiments of the present disclosure; and

FIGS. 8A-8C illustrate another example apparatus used for assessing target sequence numbers, in accordance with various embodiments of the present disclosure.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Embodiments in accordance with the present disclosure are useful for determining a copy number variation of a nucleic acid target sequence in a sample. The copy number variations between target sequences is important at the genomic nucleic acid structure level (chromosomal aneuploidy, copy number repeats within chromosomes, DNA structure, mRNA, microRNA, and other RNA targets, etc.) that vary in concentration between healthy and disease states. A specific example of a copy number variation between target sequences includes the relative concentration of chromosome 13 in a sample as compared to the concentration of chromosome 13 in a normal or healthy person. While not necessarily so limited, various aspects of the invention may be appreciated through a discussion of examples in this regard. Accordingly and in the following description, various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element.

Embodiments in accordance with the present disclosure are directed to assessing for the presence and/or concentration of a plurality of different target sequences in a sample. Somewhat surprisingly, a digital or binary result can be used to assess the concentration and/or relative concentration of a plurality of different target sequences, such as 10-10,000, at the same time (e.g., one test). To assess the plurality of target sequences, a digital technique can be used. The digital technique, as described herein, combines statistical sampling and digital techniques, and that is not sensitive to amplicon differences and/or when PCR reaction is stopped. The digital techniques can include counting the presence or absence of a target bound to unique locations of a substrate based on fluorescent signals. The substrate (e.g., a digital microarray) includes various complementary tag sequences at different (e.g., unique) locations that bind to tag sequences of probes bound to target sequences.

For example, a sample can be exposed to a plurality of probes. The probes include sequences that are complementary to a sequence in the target, which can be referred to respectively as the “complementary target sequence” (or “complementary sequence”) and the “target sequence.” The target sequences present in the sample bind to respective probes that have complementary target sequences to the target, sometimes herein referred to as “hybridization.” The number of each type of target that binds to an appropriate type of probe bears a relationship, such as but not limited to a linear relationship, to the concentration of that target in the sample. Thus low concentration genomic targets bind to the probe pool in smaller numbers compared to higher concentration targets. The probe structure experiences an inversion and circularizes, forming a loop, while hybridizing. Target sequences that do not bind to a probe (e.g., do not circularize) are removed through a target purification process, such as by adding exonuclease to the sample to remove the non-circularized DNA. In probe approaches that do not use circularization, common techniques include binding to beads and washing away unbound probes. After hybridizing to the probes, the number of bound target sequences in the sample is increased via an amplification process. The amplification process can be a PCR process that amplifies a single or few copies of the amplicons (e.g., target sequences bound to a probe) across several orders of magnitude.

A signal, such as a fluorescent signal, for each of the different locations (e.g., tag sequence locations) is read using scanning circuitry and then converted to a binary value (i.e., present or absent) based on a threshold. The number of bindings on the substrate can then be counted, similar to “yes and no” bucket counts. For example, the probes also include different tag sequences that can bind to complementary tag sequences on the substrate and which is used to detect the presence of the target sequence. A plurality of different locations of the substrate are associated with the tag sequences that are indicative of a particular target. In specific embodiments, at the end of the PCR process and hybridization process, each different (e.g., unique or discrete) location is analyzed to determine if the tag sequence is present or not (e.g., using fluorescent labels). In response to determining the tag sequence is present at a particular location, a bucket count indicative of the presence of the target is increased by one. The digital values for each tag sequence indicative of the target are summed to quantify the target concentration. The total strength of the signal, from a spot (feature) on the substrate (e.g., microarray), can depend upon the amount of target binding to the probes and the tag sequence present on that spot.

As previously described, a sample can be exposed to a plurality of probes. Exposing a sample to probes can include mixing probes with a sample, forming a mixture or solution of the probes, the sample and, optionally another a solvent, and/or other known techniques for exposing a sample to probes. As previously described, the plurality of probes include a plurality complimentary sequences that bind to the plurality of target sequences and a plurality of different tag sequences for each of the plurality of probes directed to one of the plurality of target sequences in the sample, with the different tag sequences binding to different locations on the substrate. Molecular inversion probes (MIPs) or other separate and non-inverting probes can be used for the analysis of nucleic acids using substrates having a plurality of complementary tag sequences, e.g., microarrays. In specific examples, a plurality of probes are mixed in with a sample that can contain one or several targets (e.g., sequences) that are analyzed. Although embodiments are not limited to MIPs, for ease of reference, probes are generally referred to as MIPs herein. Each MIP includes a complementary sequence that can bind with a specific target. Each MIP also has a unique tag that can hybridize to a different (e.g., unique) location on a substrate. In specific examples, the plurality of probes include a set of M-probes for each target, where each of the M-probes includes a unique tag sequence. Several sets or types of MIPs are mixed in, each set or type able to bind to a specific target sequence with each MIP containing a unique tag sequence.

The MIPs bound to the target sequence are caused to bind to different locations on the substrate. Causing MIPs to bind to different locations on the substrate can include placing the bound target sequences in contact with the substrate, washing the bound target sequences over (and in contact with the substrate), and/or depositing the bound target sequence onto the substrate, among other techniques for exposing the bound target sequences to the substrate. For example, the amplified bound target sequences are placed on and/or in the presence of the substrate (e.g., digital microarray). At least portions of the MIPs bound target sequences bind to different (e.g., unique) locations on the substrate. Specifically, the respective tag sequences of the MIPs (that are bound to a target sequence) bind to complementary tag sequences on the substrate.

The number of the target sequences present on the substrate can be assessed by using scanning circuitry and information indicative of the different locations and associated target sequence. Assessing the number of target sequences present on (e.g., indirectly bound to) the substrate can include a counting scheme and/or an output of a digital value for each the plurality of different locations on the substrate based on a determination of whether a target sequence is present at each respective different location or not. In specific embodiments, the assessment includes scanning the substrate for signal intensities indicative of target sequences present on and/or tag sequences bound to the substrate, counting a copy number of a target sequence present on the substrate and/or the number of tag sequences bound on the substrate (and associated with the target) using the signal intensities, determining copy number variants of the target sequences, quantifying a concentration or relative concentration of a target sequence in the sample, and/or comparing the copy number to a threshold indicative of a diseased or health state, among other assessment techniques described herein.

As an example, after amplification and hybridization on the substrate, a counting scheme is implemented to determine the copy number of each target sequence in the original sample. As previously describe, a plurality of unique locations are associated with a tag sequence indicative of a particular target. At the respective unique locations includes a complementary tag sequence to the respective tag sequences associated with the target, sometimes herein called “complimentary tag locations”. At the end of the amplification and hybridization processes, each unique location is analyzed to determine if the tag sequence indicative of the target is present or not (e.g., using fluorescent tags). By using information indicative of the different locations and associated tag sequences and/or with target sequences, the number of the target sequences present on the substrate are counted based on a fluorescent signal of the tag sequence in the probe bound on the substrate. In response to determining the tag sequence is present at a particular location, a count indicative of the presence of the target is increased by one and the digital values for each tag sequence indicative of the target is summed to quantify the initial target concentration, herein sometimes referred to as a “target count score”. As previously discussed, the presence of the target can be indicative of a disease and/or physiological condition. In various embodiments, a plurality of targets are analyzed and a target count score is generated for each target. The target scores are further processed, such as comparing to a threshold or threshold value that is indicative of a diseases state and/or other processing for prognosis, diagnosis and/or treatment purposes.

With this technique, small changes in a sample's initial target concentration are as detectable as large changes in sampling. Specifically, each target is assigned some number of “tag” sequences—that have minimal potential for cross hybridization. Examples of commercial tag sequences can be found on the Affymetrix TAG array. The digital results reduces the time for detection and increases the precision and sensitivity to concentrations of targets, as compared to other techniques. For example, the digital results and/or concentrations determined are not sensitive to small concentration differences, amplification differences between amplicons and/or to determining when to stop the PCR reaction. Various specific embodiments include methods of analyzing approximately 10-100,000 molecules, although embodiments are not so limited.

In various embodiments, each of the tag sequences is introduced during a molecular inversion probe ligation reaction. For example, MIPs containing the X “tag” sequences hybridize to the target sequence and ligate randomly with a probability relative to the sample's initial target concentration. The resulting distribution of unique incorporated tag sequences is therefore a representation of that sample's initial concentration. After amplification, the reaction is hybridized to a substrate (e.g., a microarray). The substrate (e.g., a microarray) is designed such that it consists of complementary sequence (e.g., DNA) features for each unique “tag” sequence. After hybridization, the “tag” probe intensities are background corrected, normalized and converted to binary (off/on as 0 and 1) values (using a simple pass/fail threshold) using processing circuitry. This thresholding reduces the impact of amplification efficiency differences between amplicons. The digital values for each target are summed to quantify the initial target concentration using the processing circuitry and can be used to quantify the target concentrations of a plurality of targets in the sample.

For reference, it should be noted that due to advances in synthetic biology a large number of unique tags pools can be created at a low cost by commercial vendors such as Twist Biosciences and CustomArray. Thus, there is no and/or mitigated limitation posed by the number of unique tags that are needed.

Further, while the embodiments above describe the use of MIPs, other probes or ligation assays may also be used instead of the MIPs. One such example is the digital analysis of selected regions (DANSR) assays. The embodiments described in this disclosure apply to various types of assays.

Embodiments in accordance with the present disclosure convert what is often an imprecise analog readout approach to a highly-reliable precise digital readout, allowing for detection of small changes in copy number. Digital readout is advantageous as compared to analog readouts as the digital readout significantly lowers production cost. The approach described above enables the precision of digital readout and the cost of microarrays. Further, the digital microarray readout can mitigate the effects of concentration changes caused by PCR biases between amplicon sequences.

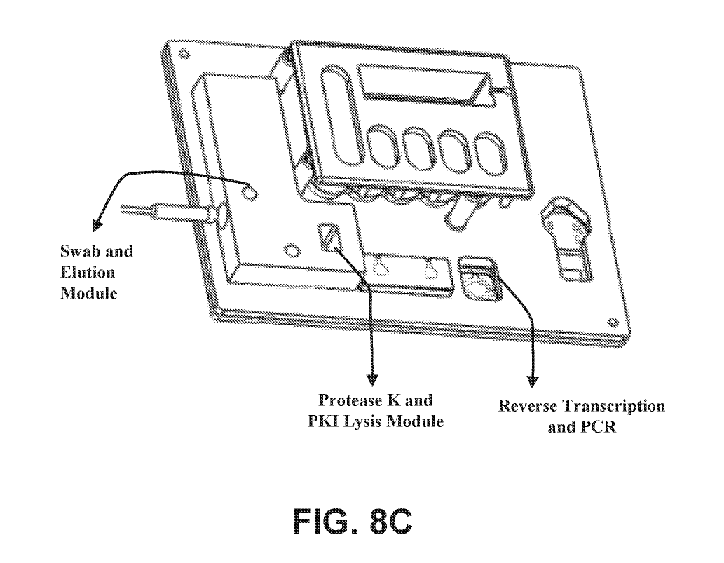

In some specific embodiments, the digital output is implemented using one or more apparatuses. The apparatus includes processing circuitry and scanning circuitry. The scanning circuitry is used to capture fluorescent signal intensities indicative of tag sequences bound to the substrate (e.g., a surface of the microarray). The processing circuitry uses the captured fluorescent signal intensities to provide the digital output. The apparatus can additionally include a microfluidic card with a plurality of chambers that are in fluidic connection and that are used to perform the hybridization of the probes to the targets in the sample, purification, and amplification, such as the rapid assay apparatus as further illustrated herein by FIGS. 8A-8C and as further illustrated by page 2 of the underlying Provisional Application entitled “Rapid Assay Process Development”, and illustrated on page 2 of the attached appendix of the underlying Provisional Application entitled “Digital Microassay” as above-mentioned (which characterizes examples in text and a number of the figures as corresponding with the embodiments discussed in this section of the present disclosure). In some embodiments, the apparatus can also perform the function of hybridization of the amplicons to the substrate. In such an apparatus/microfluidic card, relevant chambers and/or modules (as illustrated by FIGS. 8A-8C, as well as at page 2 of underlying Provisional Application entitled and page 2 of the Appendix of the underlying Provisional Application) are in fluidic communication so as to pass the sample from one chamber/module to the next for operating on the sample according to the functionality relevant thereto, such as the hybridization to probes, target purification, and amplification. In other embodiments, one or more additional apparatuses as used to perform the hybridization and amplification processes, such as various thermal cyclers.

The digital technique is implemented for diagnostics and/or treatment determinations that involve determining copy number variations between normal and diseased states. A variety of disease states and/or physiological conditions result in copy number variations in different nucleic acid biomarkers as compared to a normal state (e.g., a person that does not have the disease). While not limiting, examples of nucleic acid copy numbers variations can be found in multiple copies of entire chromosomes, multiple copies of specific genes within a chromosome, differential transcription of protein coding sequences (e.g., mRNA), and non-coding sequences (e.g., microRNA). Further, various embodiments include the analysis of circular RNAs, and small non coding RNA to detect nucleic acid using the digital microarray technology (using the discovered nucleic acid biomarker classes).

A substrate (e.g., a digital microarray) can be used to provide a digital readout of chromosome number status of a person. The typical human cell has 46 total chromosomes, however certain conditions are associated with extra (trisomy as opposed to diploid) chromosomes. The most common example is Trisomy 21 (aka, Down's Syndrome), other viable conditions are Trisomy 12, 18, X and Y. The digital readout using the digital microarray is both precise and cost efficient.

Various embodiments include a readout of sequence amplification within a single chromosome. An example of the diagnostic value of within chromosomes sequence amplification is the human epidermal growth factor receptor 2 (HER2) gene. The HER2 gene has been implicated in approximately twenty-five percent of breast cancer diagnoses. Fluorescence In Situ Hybridization (FISH) can be used to determine the number of HER2 gene copies in a cancer cell. The copy number status of a tumor can be useful to the effectiveness of treatment approaches. For example, a number of drugs (e.g., Herceptin, Perjeta and Tykerb) are used to treat tumors that have an overexpression of the HER2 gene.

In addition to chromosomal changes, the expression pattern of genes transcribed into mRNA can have implications in human disease states. For example, the transcription status of genes in the form of mRNA are used to guide treatment of determine prognosis. In various embodiments, the digital and/or microarray technology allows for numerous mRNA copies to be precisely measured to guide treatment and prognosis.

Smaller non-protein coding RNA biomarkers, called microRNAs, can be analyzed for copy number. As with the mRNA approach, digital and/or microarray technology allows for precise counting of the number of miRNAs in a panel (around 100 different miRNA) present in a sample.

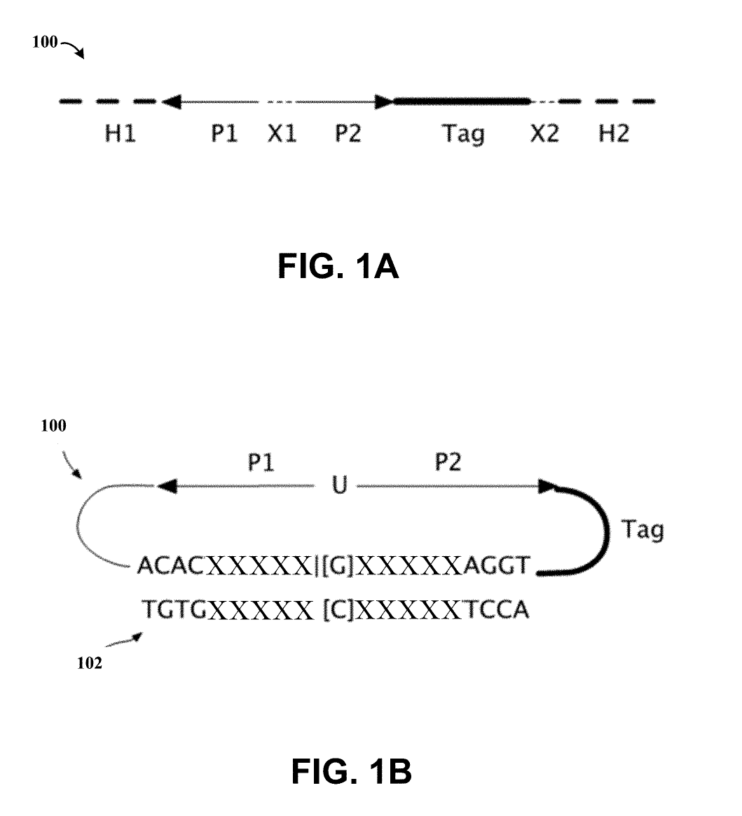

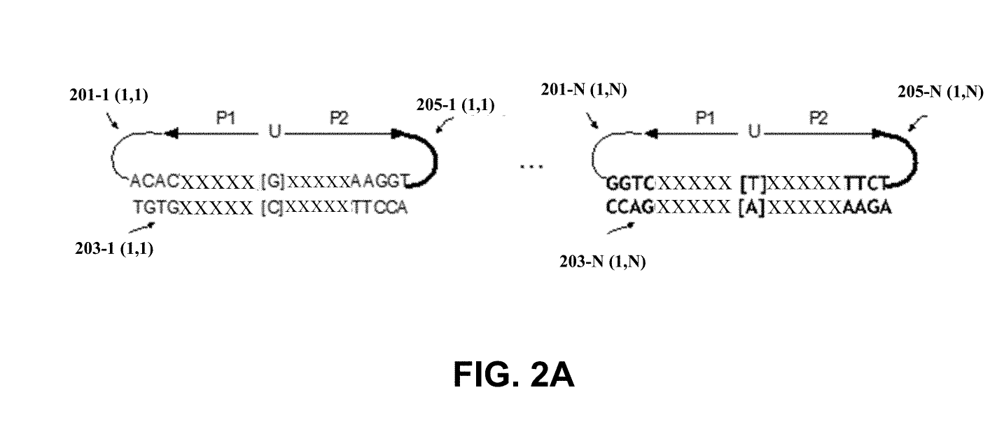

Turning now to the figures, FIG. 1A illustrates a structure of an unreacted MIP 100 in accordance with various embodiments of the present disclosure. P1 and P2 denote PCR primer 1 and PCR primer 2 (forward and reverse), respectively. The tag sequence (e.g., identified as “tag”) is a sequence of molecules that helps identify the captured target. The tag sequence is described further herein, but typically includes a different (e.g., unique) fluorescent component (e.g., a label sometimes called a “tag”) that is utilized during the detection stage. The fluorescent label, in specific embodiments, is incorporated during PCR (and is not part of the probe). H1 and H2 are two regions consisting of sequences that are complementary to sequences in the target. X1 and X2 are cleavage sites.

As further illustrated below by FIG. 1B, these probes (e.g., MIPS) start out as single stranded DNA molecules containing sequences that are complementary to the target in the genome. During the analysis process, these probes hybridize to the target, thereby capturing the target. The probe structure experiences an inversion and circularizes, forming a loop, while hybridizing. Thus, in such embodiments, the steps for analysis of nucleic acids include annealing to the target, optional gap filling—polymerization, ligation, exonuclease selection, probe release, amplification, hybridization on the microarray followed by detection.

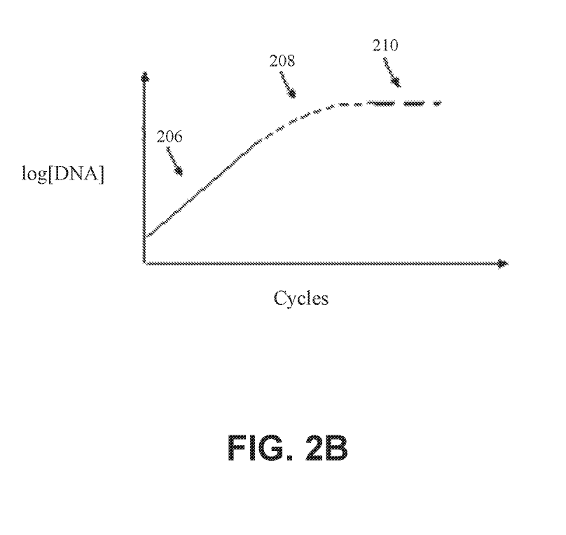

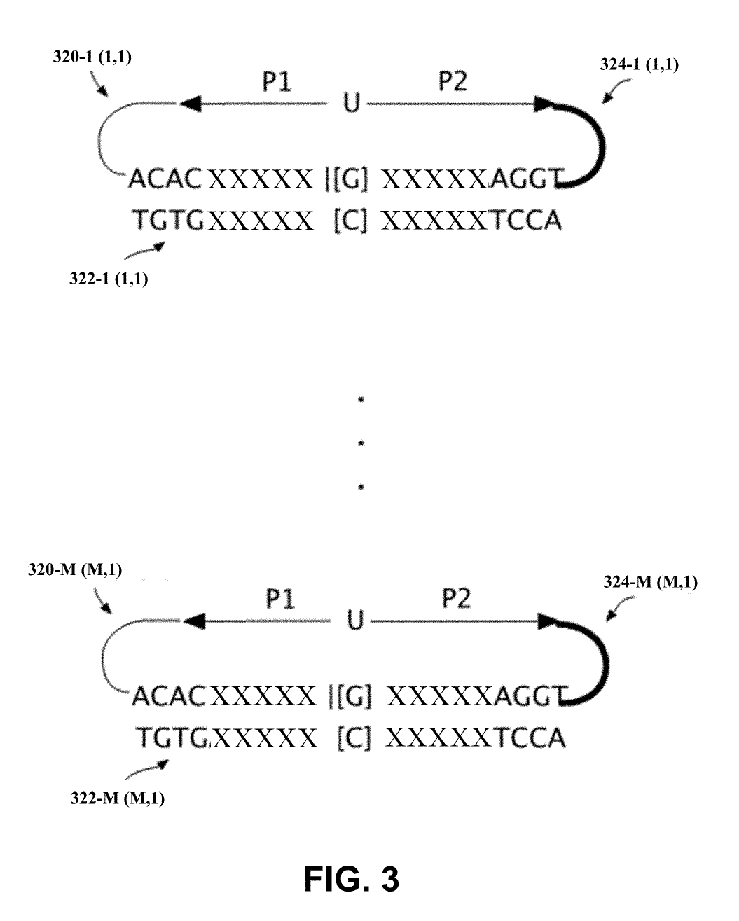

FIG. 1B illustrates an example of a MIP 100 that is circularized and bound to a target sequence, in accordance with various embodiments of the present disclosure. In various embodiments, the U stands for a uracil molecule and acts as a cleavage site. The H1 and H2 regions of FIG. 1A are replaced by example sequences in FIG. 1B. The ligation location is indicated by the symbol “|”. The genomic target sequence 102 is bound to the complementary target sequence on the probe (e.g., MIP 100). The use of tag sequence and substrates (e.g., microarrays) is further described in FIGS. 2A and 2B.

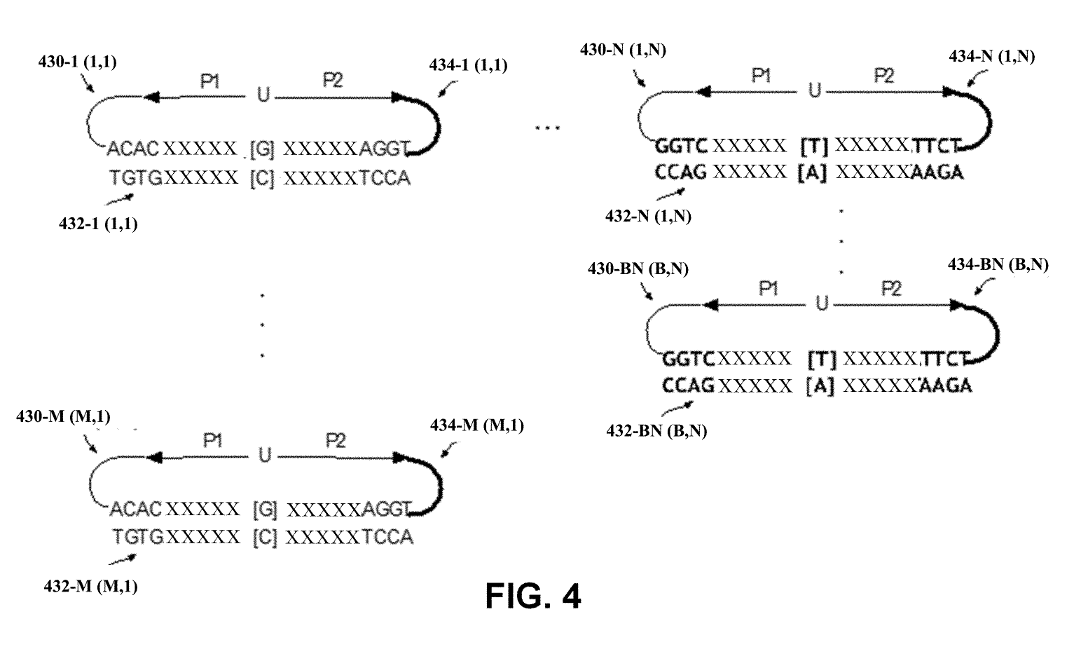

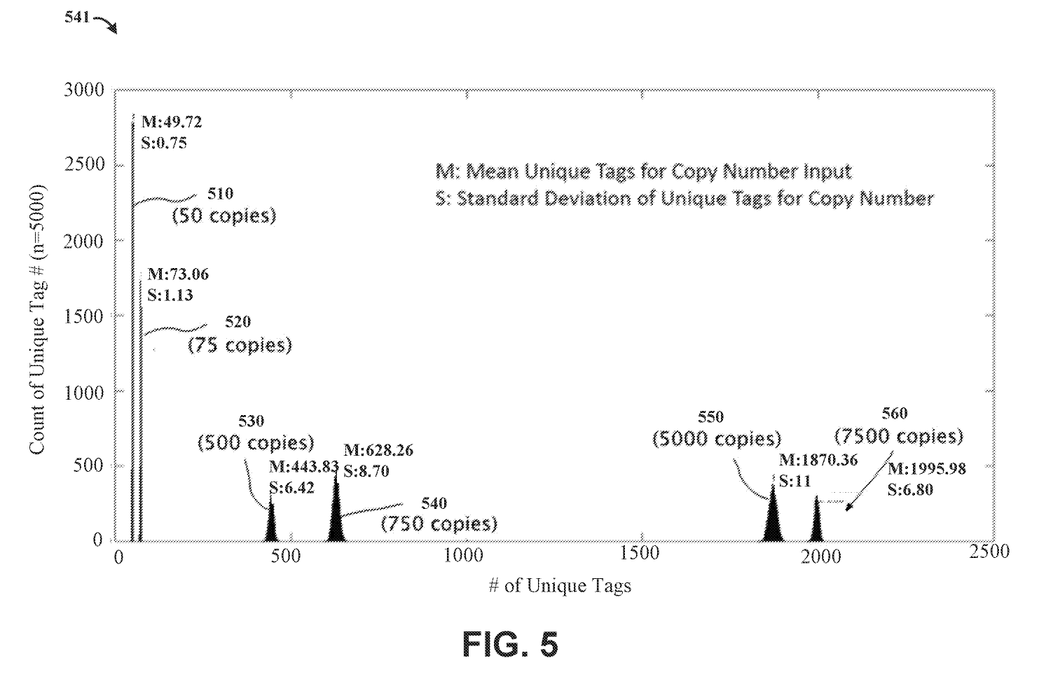

FIG. 2A illustrates N different genomic (target) sequences in accordance with various embodiments of the present disclosure. For example, the two target sequences are explicitly shown and are labeled as 203-1 (1,1) and 203-N (1,N). Each of these target sequences is bound to a MIP with a different tag sequence (e.g., a unique tag sequence). In the figure, two MIPs are shown and are labeled as 201-1 (1,1) and 201-N (1,N), depicting that there are N MIPs. Each MIP incorporates a different tag sequence. In specific embodiments, the different tag sequences can each include a unique tag sequences. The different (e.g., unique) tag sequences refer to or include nucleic acid sequences that bind to different locations on the substrate via complementary tag sequences at the locations, also referred to as complementary tag locations. Thus, there are N different tag sequences (e.g., unique tags), two of which are shown and labeled as 205-1 (1,1) and 205-2 (1,N). As may be appreciated and further described herein, the representation of “(1,1)” and (1,N)” in FIG. 2A (as well as FIGS. 3-4), the x value (e.g., 1 or 1) is indicative of the target sequence and the y value (e.g., 1 or N) is indicative of the probe and/or the tag sequence associated with the target sequence. After amplification, each amplicon hybridizes to a particular (e.g., unique) location on the substrate that is complementary to the tag sequence in the MIP. With the information about the location on the substrate, the genomic targets are identified. The strength of the fluorescent signal may also provide qualitative or semi-quantitative data about the concentration of specific genomic targets in the sample.

FIG. 2B illustrates a typical curve relating the concentration of a DNA molecule to the number of PCR cycles in accordance with various embodiments of the present disclosure. At the initial stages (e.g., region 206) of the PCR cycle, the concentration increases exponentially and the relationship between the log concentration and cycles is approximately linear. Later, in the region 208, the concentration increases linearly until it plateaus out in the region 210. Traditional PCR detection (or end-point detection) is carried out in region 210. Real-time PCR is carried out in the region 206. Both these and other techniques rely on the concentration rates. However, when the concentration has plateaued out, the relationship between the initial concentration and the plateaued concentration is lost. To avoid this, analysis is carried out in regions 206 and 210 but that then depends on having sufficient knowledge about the relationship between the concentration and cycles. Further, each molecule may have a different relationship between concentration and cycles and if multiple molecules have to be analyzed, it may be quite difficult to determine when to stop the PCR reaction. These disadvantages are overcome in the method described below.

FIG. 3 illustrates an example process of using tags to determine concentration of a single target in accordance with various embodiments of the present disclosure. The figures illustrate M copies of the same target sequence, two of which are labeled as 322-1 (1,1) and 322-M (M,1). The complementary target sequences on the MIPs that bind to the target are also the same. However, the tag sequences are unique—there are M different (e.g., unique) tags, two of which are labeled as 324-1 (1,1) and 324-M (M,1). Thus, whereas in FIG. 2A, different targets are analyzed, in FIG. 3, the same target is analyzed. However in both situations, each MIP has a different tag sequence (e.g., a unique tag). Similarly to FIG. 2A, the reacted probes 320-1 (1,1) and 320-M (M, 1) hybridize with M different complementary tag sequences (e.g., the tag sequence 324-1, 324-M of the probe binds to the complementary tag sequence at the particular (e.g., unique) location of the substrate) that are in different (e.g., unique) locations on the substrate having the plurality of complementary tag sequences, e.g., a microarray. In an ideal situation when every copy of a particular target sequence binds to a probe, the copy number is determined in absolute terms. In non-ideal conditions, the copy number is determined in relative terms. In various embodiments, this process is used to determine the concentrations in a real measurement situation, as further described herein. It can also be used when multiple sequences are analyzed at the same time, as further described herein. These concepts are further described below.

FIG. 4 illustrates an example process of using tags to determine concentration of N different targets in accordance with various embodiments of the present disclosure. The N different targets (e.g., sequences) exist in a single sample. This is indicated in the horizontal direction in each row.

As illustrated by FIG. 4, the genomic (target) sequence 432-1 (1,1) is different than the genomic (target) sequence 432-N (1,N). M different tag sequences can be used to assess a concentration or relative concentration of the target sequence in the sample. For example, M different probes having M different tag sequences are used to assess the genomic sequence represented by 432-1. The M^{th} copy of the genomic sequence is indicated in FIG. 4 as 432-M (M,1). Each target sequence can exist with different concentrations and can be assessed using different sized sets of probes (e.g., the number of probes in a set can be different and/or the same per target sequence). Each target, thereby, has a set of different tag sequences associated with it (and the different tag sequences are part of a set of probes having complementary target sequences that bind to the respective target). In some embodiments, target sequence 432-N (1,N) is suspect to have B copies and/or is otherwise assessed using B different probes having B different tag sequences (e.g., 434-N . . . 434-BN). The B^{th} copy is illustrated as target sequence 432-BN (B,N). B can be equal to or different than M. In an ideal scenario, each target (e.g., every unique genomic sequence and all its copies) is captured by the probes 430-1 (1,1), 430-M (M−1), 430-N (1,N) . . . 430-BN (B,N), which can be MIPs. Each copy of these bound genomic sequences (e.g., targets) are amplified. After amplification, the amplicons are hybridized to locations on the substrate (e.g., unique locations on the microarray). As previously described, since in this ideal situation, every one of the genomic sequences and all its copies are captured, to determine the concentration of each target sequence, the number of occurrences of that specific target sequence are counted on the substrate via detecting a (binary pass/fail) presence of a tag sequence indicative of or otherwise associated with the target sequence and summing the number of detected tags for each target being analyzed. This counting is performed when the reactions have reached the plateau stage. As the counting is performed on the plateau stage, it is not necessary to track of the reaction process during the PCR cycles. Accordingly, various embodiments increase ease of use and improved precision of copy number measurements.

The digital technique can be utilized, in accordance with various embodiments, when ideal conditions do not exist. In some embodiments, not all the copies of each target sequence binds to a probe. To mitigate target copies not binding to a probe (e.g., to ensure that almost all the targets bind to a probe), an abundance of probes are added to drive each reaction.

As a specific experimental example, three unique genomic targets SA, SB and SC, are assumed present in one sample. A further assumption is made that 1000 copies of SA, 500 copies of SB and 100 copies of SC are present in this sample. Three types of probes are added to the sample MA, MB and MC, which bind to SA, SB and SC respectively. Further, in the example there are N=3 targets and each probe has M unique tag sequences, where M could between 10 and 100,000. An abundance of each probe type is added; thus in this example 1*10̂6 copies of each tag sequence variant of MA, MB and MC is added. As previously described, the probes are uniquely distinguishable as they each have a different and/or unique tag sequence and can hybridize to a particular and/or unique location on the substrate (e.g., specific locations that are known based on the design of the microarray or complementary tag sequences).

In the ideal circumstance all 1000 copies of SA, 500 copies of SB and 100 copies of SC bind to an appropriate probe. In some instances, a percentage less than 100% of the targets bind. However, due to the abundance of the each type of probe, a large percent of each target bind to the probe. For example, if 98% of the target copies bind, then 980 copies of SA, 490 copies of SB and 245 copies of SC successfully bind to appropriate probes. After PCR amplification, the 980 copies of SA, 490 copies of SB and 245 copies of SC may exist in very large numbers—however the end point concentration is not used to determine the initial concentration as each of these copies hybridizes to a particular and/or unique location on the substrate (e.g., in the microarray). For example, there may be 980 unique locations where MA is detected of the M locations for unique MA tag sequences, 490 unique locations where MB is detected of the M locations for unique MB tag sequences and 245 unique locations where MC is detected of the M locations for MC tag sequences. The tag sequences that are amplified are randomly determined, so it is only the number of tag sequences, and not the specific tag, that is detected above background. The 98% number above is used as an example—other percentages are possible. However as stated above, due to the abundance of probes a large percentage close to 100%, of target sequences are expected to react. With this method, the relative concentration of the various unique sequences can be determined. Due to the abundance approach, the relative concentration is a good approximation of the actual approximation.

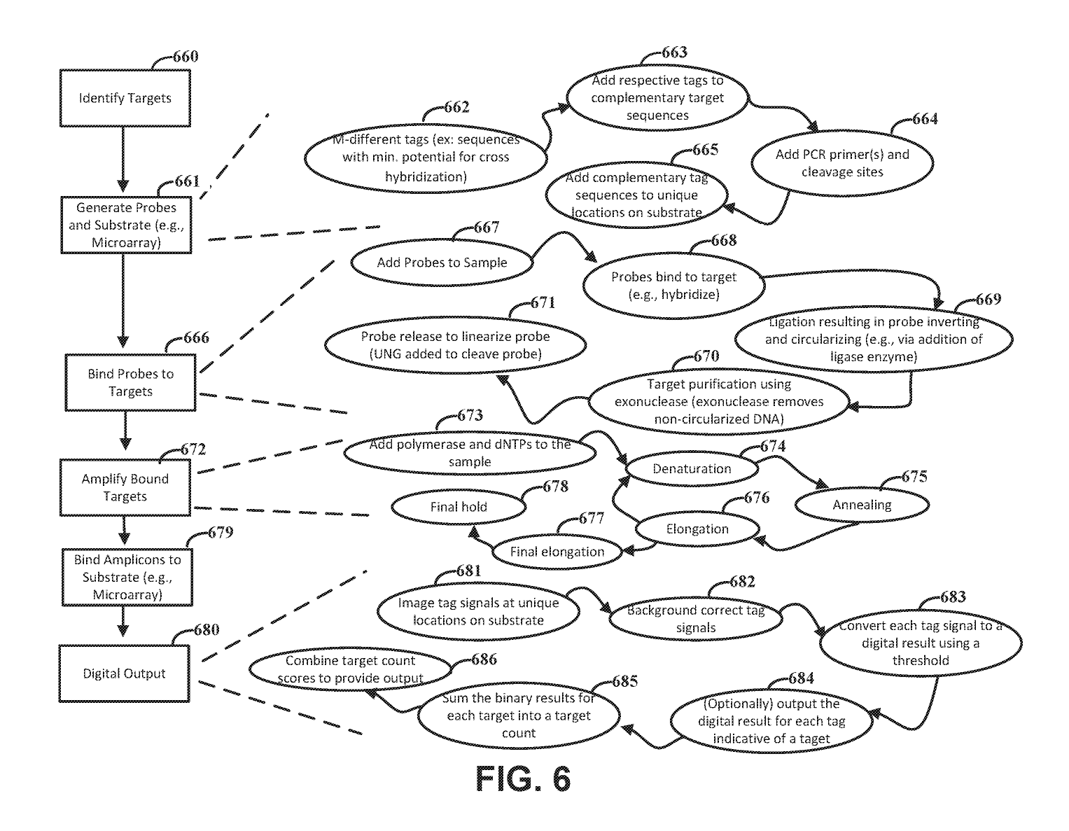

In accordance with various embodiments of the present disclosure, experimental results have been demonstrated to evidence the surprising results that a microarray provides a presence and/or relative concentration of targets in the sample as a digital result that is precise and efficient. As illustrated in connection with FIG. 5 (as discussed further below), such results can be readily modeled and/or simulated for a situation where 50 copies of genomic target sequence 510, 75 copies of genomic target sequence 520, 500 copies of genomic target sequence 530, 750 copies of genomic target sequence 540, 5000 copies of genomic target sequence 550 and 7500 copies of genomic target sequence are present in a sample. In such experimental embodiments, 2048 different (e.g., unique) probes are added to the sample that are capable of binding to each of the genomic target sequences listed above. Accordingly, the presence of and/or relative concentrations of the target sequences in the sample can be readily identified by the number of probes present on the surface of a microarray which are each provided as a digital output (present or not present). The digital outputs of a target sequence (e.g., each tag sequence associated with a target sequence) are summed to efficiently and precisely provide the relative concentration and can be used to concurrently provide concentrations of multiple target sequences in a single sample.

Statistical analysis further demonstrates that the presence and/or relative concentrations of the sequences in a sample can be readily identified in accordance with the various embodiments presented in the instant disclosure. As an example, reference may be made to the histogram 541 of FIG. 5 which statistically shows of the number of unique tags that are counted after the tags (in an experimental embodiment) are circularized, amplified and hybridized to the microarray after 5000 runs of the experimental embodiment. For target sequence 510, which has 50 copies present in the sample, the experimental embodiment shows that after 5000 runs, the different (e.g., unique) tag sequences associated with target sequence 510 are counted with a mean of 49.72 with a standard deviation of 0.75. Similarly for target sequence 540 which has 750 copies present in the sample, the experimental embodiment shows that after 5000 runs, the different (e.g., unique) tag sequences associated with target sequence 540 are counted with a mean of 628.26 with a standard deviation of 8.70. As can be seen, the number of tag sequences associated with target sequences 510, 520, 530 and 540 are close to the actual number of copies present. However, for target sequence 550 and 560, where 5000 and 7500 copies are present, the number of probes (2048) is not enough. Thus, the mean number of unique tag sequences for target sequence 550 for example is 1870.36 with a standard deviation of 11. Even with this scenario, it can be seen that target sequences 550 and 560 are distinguishable.

For example, assume the number of copies of each target sequence in the above-described sample is unknown. MIPs that contain probes configured to bind to target sequences and unique tag sequences are added to the sample. The number of MIPs added is the same number for each target and/or different for each or at least two or more targets. In some specific examples, the probes are designed to bind to target sequences that are known to be indicative of a disease and/or of a particular chromosomal abnormality. As a specific example, the MIPs added are indicative of four different diseases that a fetus is being tested for. In other embodiments, the MIPs added are indicative of different sequences of a specific cancer. As previously discussed, the probe of the MIP is a complementary sequence to a target sequence. Once the MIPs are added to the sample, if one or more of the sequences in the sample is the target sequence, the probe of the MIP binds to a copy of the respective target sequence. For example, if each of the target sequences 510, 520, 530 and 540 in the sample is a target, the MIPs added bind to copies of the target sequences 510, 520, 530 and 540.

Several sets of MIPs can be mixed with the sample, each set configured to bind to a target and each MIP set including a plurality of different tag sequences (e.g., unique tags) configured to bind to different locations of the microarray (e.g., unique locations on a surface of the microarray or complementary tag locations). MIPs bound to complementary target sequences present in the sample are amplified and hybridized on the surface of the microarray at the different locations (e.g., on the microarray at the unique locations). The number of hybridizations are counted to determine the relative copy number of each target in the sample. For example, during hybridization, the MIPs that have X “tag” sequences hybridize and ligate randomly with a probability that is relative to the sample's initial target concentration. The number of the target sequences 510, 520, 530 and 540 that bind to a MIP bears a relationship to the copy number of the target sequences. For example, the target sequence 510 which has 50 copies has a lower concentration of being bound to a MIP than the target sequence 550 which has 5000 copies. Thereby, the target sequence 510 hybridizes and ligates on the microarray at a lower concentration than the target sequence 550.

After amplification, each amplicon hybridizes to a particular location on the microarray (e.g., a unique location on the microarray) that includes a complementary tag sequence to the tag sequence in the respective MIP. Sequences or material that does not bind to the microarray are removed such via a washing technique. With information about the microarray, such as knowledge of complementary tag sequences of the different (e.g., unique) locations and/or complementary tag sequences that are associated or indicative of a target sequence, the presence or absence of the tag sequence indicative of or associated with a target is identified using processing circuitry and scanning circuitry (e.g., microarray scanning circuitry). For example, a digital “present or not” is output for each tag sequence (e.g., at the unique locations of the complementary tag sequences). Specifically, various locations are associated with different targets and/or different tag sequences. The strength of the fluorescent signal, as captured by the scanning circuitry, can be used by the processing circuitry to provide semi-quantitative data above the concentration of the specific targets in the sample, at least relative to one another. For example, the number of hybridizations for a target sequence is counted based on knowledge of the complements of the locations on the microarray. As a specific example, if target sequence 540 (which has 750 copies) is associated with the number of copies of chromosome 13, the hybridization of target sequence 540 indicates the presence of three copies of chromosome 13 (e.g., Trisomy 13).

To provide the digital output, the tag sequence intensities can be background corrected, normalized, and then converted to a binary (e.g., digital) result, such as “off/on” or “pass/fail” values, using a threshold using the processing circuitry. The background correction, in various embodiments, includes a background noise value that is indicative of background (e.g., noise that is not a signal). For example, when no probes bind to the substrate (e.g., microarray), some fluorescent signal is detected, even though no tag sequence is present. The signal detected, when no tag sequence is present/bound, to the substrate is background noise. The detected signal is corrected (e.g., the background noise value is subtracted from the fluorescent signal intensity) based on the background noise value. Further, the threshold includes a signal value that is considered pass or fail. For example, and purely for illustrative purposes, the background noise value is 10 with a standard deviation of 5. A signal is received that is 35. The background noise value is removed from the signal to give a background corrected value of 25. The threshold includes 35. Because the background corrected value is not greater than the threshold, the binary result of the tag sequence that corresponds to the signal is a “0” or a “fail”. The thresholding reduces the impact of amplification efficiency differences between amplicons.

The binary results are counted for each tag sequence indicative of a target and for each target. For example, assume two targets are being analyzed and each target has one-thousand tags. Each target has one-thousand binary results that are counted and summed to provide a target count score. Using the above example, two target count scores are provided.

In some embodiments, the target count scores are further processed. For example, another function is performed on the target count scores to provide prognosis, diagnosis, and/or treatment information. The further processing can include a threshold for the target count scores that are based on expected results (e.g., numbers) for a person that does not have a disease or other physiological disorder associated with the target, experimental results, and/or based on reference information. Using the above-provided example of Trisomy 13, when testing maternal blood to determine if the fetus has Trisomy 13, a particular concentration or quasi-concentration of chromosome 13 indicates that the fetus has or does not have Trisomy 13. The digital value for each tag sequence indicative of a chromosome 13 is summed to quantify the initial target concentration as a target count score and the target count score is compared to the threshold. However, embodiments are not so limited and in some embodiments the further processing includes comparing the target count score and/or the combined target count scores for each target to background information that is indicative of a prognosis (e.g., likelihood of surviving five years, ten years, and fifteen years), diagnosis, and/or treatment. As a particular example, certain cancer cells respond to different drugs with greater effect.

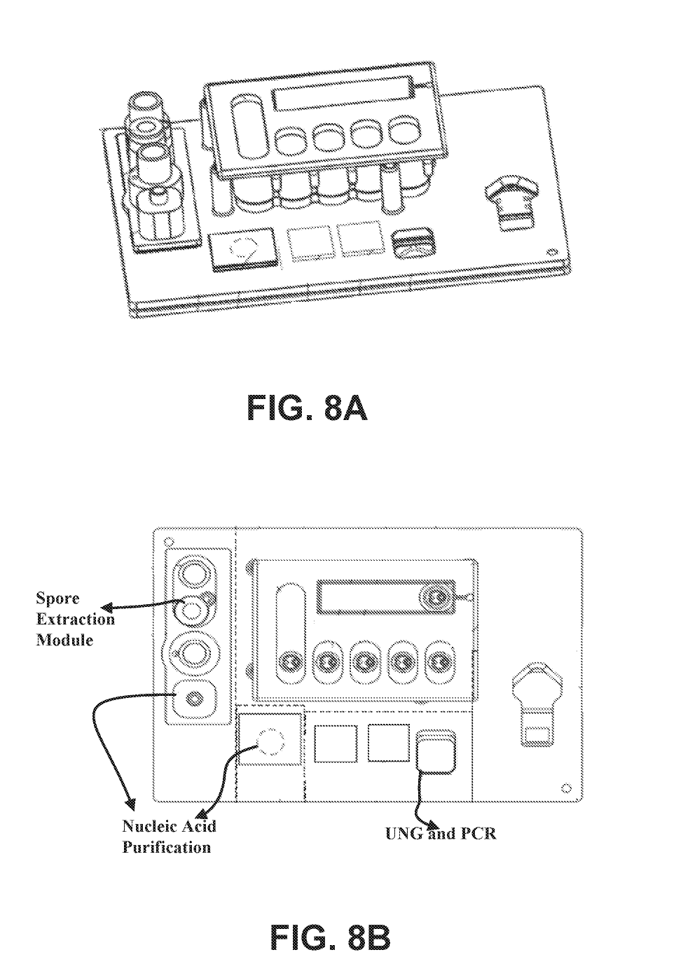

Related embodiments include or are directed to a rapid assay apparatus and/or non-invasive pregnancy testing (NIPT) as described with respect to FIG. 7 and FIGS. 8A-8C, page 2 of the underlying Provisional Application entitled titled “Rapid Assay Process Development”, and page 2 of the attached appendix of the underlying Provisional Application entitled titled “Digital Microassay”.

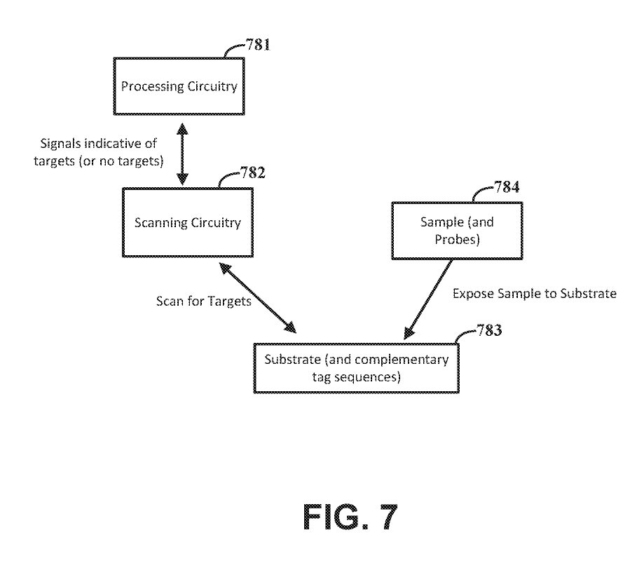

FIG. 6 illustrates an example process for providing a digital result for a disease or condition using a substrate, in accordance with various embodiments of the present disclosure. In some embodiments, the process is used to provide a digital output (e.g., binary result such as “pass” or “fail”) for one or more diseases or conditions. In other instances, the process is used to identify copy number variations between chromosomes and/or sequences.

At 660, one or more target sequences are identified. The identification is based on the particular test being performed. For example, if a non-invasive pregnancy test (NIPT) is being performed, one or more genetic disorders to test for are identified. In some embodiments, one target sequence is analyzed and, in other embodiments, a plurality of target sequences are analyzed (e.g., 100-1000 targets or 10-10,000). The specific target sequence can be identified using reference information, such as a database containing known and/or suspected nucleic acid sequences associated with a target.

At 661, the probes having a plurality of tag sequences and a substrate having a plurality of sequences complementary to those tag sequences are generated (e.g., designed) based on the one or more targets. The probes for a given target sequence can include MIPs, as illustrated in FIG. 1A, that include a complementary sequence to bind to the target sequence and a unique tag sequence (e.g., a fluorescent label is later incorporated during PCR). The substrate (e.g., a microarray), as previously discussed, includes the complementary tag sequences at a plurality of different locations, such as unique locations or complementary tag locations.

In specific embodiments, the probes and substrate, e.g., microarray, are generated by obtaining or creating M-different tag sequences for each of the one or more targets, at 662, where M can be different for each target. For example, a plurality of probes can be generated that contain M-different tag sequences for each target, and were the tag sequences of the plurality of probes (all of the tag sequences) have minimal potential for cross hybridization. Further, all complementary tag sequences on the substrate are designed for minimal potential for cross hybridization. In various embodiments, the tag sequences are obtained from a commercial provider. At 663, the respective tag sequences are added/combined to the sequences that are complementary to target sequences (e.g., the complementary target sequence of the probes). Further, at 664, PCR primer(s) (e.g., forward and backward primer P1 and P2 as illustrated by FIG. 1A) and cleavage sites are added to the probes. The generation of probes can include generating a set of probes with M-different tag sequences for each of the one or more targets. Specifically, the plurality of probes can include a set of probes for each target. A set of probes for a particular target includes a plurality of complimentary sequences that bind to the target sequence, and a plurality of different tag sequences that bind to a particular location of the plurality of different locations on the substrate (e.g., a set of probes, where each probe in the set includes a copy of the complementary target sequence and one of the plurality of different tag sequences). At 665, the complementary tag sequences are added to the plurality of different locations on a surface of the substrate (e.g., unique locations of the microarray), such as by using spotting techniques. For example, a microarray can be generated by spotting the complementary tag sequences at the plurality of different locations on a surface of the substrate, and forming complementary tag locations on the substrate.

At 666, the probes bind to the respective target sequence. For example, at 667, the probes are added to the sample and, at 668, bind to respective target sequences (e.g., hybridize). In some specific embodiments, MIPs can bind to a target circularize via a ligation process. At 669, a ligase enzyme is added to the sample that causes the bound targets and MIPS to circularize. Further, at 670, a target purification process is performed to remove the non-bound sequences. For example, exonuclease ii added to the sample to remove non-circularized sequences. Uracil-DNA glycosylase (UNG) can be added to the sample to cleave the cleavage site of the probe to linearize the bound target, at 671.

The number of bound targets is increased via an amplification process, at 672, although examples are not limited to the PCR process illustrated by FIG. 6 and can include a variety of PCR processed. The bound targets can be amplified via a PCR process using the universal PCR primers (P1 and P2). As a specific example of a PCR process, at 673, the enzyme polymerase and deoxynucleoside trisphosphates (dNTPs) are added to the sample. Polymerase, such as Taq polymerase, is an enzyme that synthesizes nucleic acid molecules from deoxyribonucleotides. The dNTPs are the building blocks, e.g., the deoxyribonucleotides, from which polymerase synthesizes new DNA and/or RNA strands. Other components and reagents may be added, such as a buffer solution to provide a chemical environment that is suitable for activity and stability of polymerase, bivalent cations, magnesium, manganese ions, and/or potassium ions. In various embodiments, the various components and/or reagents are added to the sample via movement of the sample through one or more chambers of a microfluidic card, such as a rapid assay apparatus, although embodiments are not so limited and can include the addition of components and/or reagents through other techniques.

The example PCR process includes repeated cycles of temperature changes. The cycling includes denaturation, at 674, annealing, at 675, and elongation, at 676. Denaturing can include heating the reaction to a first threshold temperature (e.g., 94-98 degrees Celsius) for a period of time, such as 20-30 seconds. Such denaturing causes nucleic acid melting by disrupting the hydrogen bonds between complementary bases and results in single-stranded nucleic acid molecules. The annealing operation can include heating the reaction to a second threshold temperature that is lower than the first threshold temperature (e.g., 50-65 degrees Celsius) for a period of time, such as 20-40 seconds. Such annealing causes the PCR primers binding (e.g., anneal or hybridize) to the target. The elongation can include heating the reaction to a third threshold temperature which is dependent on the particular polymerase used, whether Taq polymerase or another suitable thermostable DNA polymerase. Using Taq, this polymerase has optimum active at a temperature of 75-80 degrees Celsius and a temperature of 72 degrees may be used. During the elongation process, polymerase synthesizes a new nucleic acid strand complementary to the target by adding dNTPs that are complementary to the target in 5′ to 3′ direction, and condenses the 5′-phosphate group of the dNTPs with the 3′-hydroxyl group at the end of the nascent (extending) nucleic acid strand.

After the repeated cycles, at 677, a final elongation is performed. The final elongation includes heating the reaction to a fourth threshold temperature (e.g., 70-74 degrees or a value less than 90 degrees Celsius) for a period of time, such as 5-15 minutes. The final elongation process is used to ensure any remaining single-stranded nucleic acid sequence is fully extended. Optionally, after the final elongation, at 678, a final hold is performed. The final hold includes cooling the reaction to a particular temperature (e.g., 4-15 degrees Celsius). In various embodiments, the amplified reaction is stored at the particular temperature. In other specific embodiments, the amplicons are not stored but rather analyzed immediately after the amplification process.

At 679, the amplicons are bound to the substrate, such as a digital microarray. For instance, the amplicons (e.g., amplified probe sequences) are placed on the digital microarray. In response, target sequences indirectly bind to unique locations on the microarray by the respective tag sequences (of probes bound to the target sequence) binding with complementary tag sequences on the microarray.

At 680, a digital output is provided by analyzing the surface of the substrate. For example, at 681, fluorescent signals at the unique locations of the substrate, and indicative of a tag sequence and associated target, are analyzed and/or imaged using scanning circuitry. The fluorescent signals are referred to as tag signals in FIG. 6. In response to detecting a tag signal, at 682, the intensity of the tag signal is background corrected using a background noise value and normalized. In various embodiments, at 683, the background corrected and normalized tag signal is compared to a threshold to convert the output to a digital result (e.g., 0 or 1, pass/fail, off/on) for each tag indicative of a target. The threshold includes a simple pass/fail threshold, as previously discussed. Optional, at 684, the binary result is output for each unique location that is associated with a tag sequence indicative of a target being analyzed.

The digital results (e.g., counts) of each tag sequence indicative of or otherwise associated with a target are summed to provide a target count score, at 685. For example, each pass or “1” of tag sequences indicative of the target are summed. The target count score is indicative of the initial concentration of the input sample. In various embodiment, at 686, the target count scores, alone or in combination, are further processed to provide a diagnosis, treatment, and/or prognosis output. For example, the targets being analyzed can be indicative of cancer cells and healthy cells. A combination of the target count scores are used to output information on prognosis of the user (e.g., likelihood of survival and/or length of time). In other embodiments, a single target count score and/or a combination of target count scores is used to generate a treatment plan, such as particular drugs to provide the user.

As illustrated by FIG. 6, the digital (microarray) output is provided by outputting a binary pass/fail for each tag indicative of a target. For example, the below table summarizes an example of an output from a digital (microarray) technique:

| Target 1 | Target X |
|---|---|
| Tag 1 = 0 | Tag 1 = 1 |
| Tag 2 = 1 | Tag 2 = 1 |
| Tag 3 = 1 | Tag 3 = 1 |
| Tag 4 = 0 | Tag 4 = 0 |
| . . . | . . . |
| Tag Y = 0 | Tag Y = 0 |

As illustrated, the analysis is of X targets and each of the X targets has Y tags. Further, each of the Y tag has a binary output of “0” or “1”. The output “1” results for a target are summed to provide a target count score for each target being analyzed. For example, Target 1 has a target count score of 50 and Target X has a target count score of 75. The target count scores are indicative of the initial concentration of the target in the sample (e.g., quantification of how much Target 1 and Target X are present in the input sample). And, using the target count scores, diagnosis, treatment and/or prognosis information (e.g., to provide “meaning”) is output by further processing the target count scores using a database and/or other information. The above-illustrated table is for discussion purposes only and embodiments in accordance with the present disclosure are not limited to use of such a table.

As demonstrated and appreciated by a skilled artisan in view of the present disclosure, FIG. 6 illustrates one example process which can be applied across a variety of applications. These include, as examples and without limitation, determining copy number variations between normal and diseased states, chromosome number status of a person, sequence amplification within a single chromosome, and expression patterns of genes transcribed into mRNA. Specific examples of the above include inter alia, Trisomy 21, Trisomy 12, Trisomy 18, Trisomy X, Trisomy Y, copy number of HER2 gene in a cancer cell.

In accordance and consistent with the instant disclosure, another specific example uses HER2 gene with the specific target sequence of the HER2 gene being used to generate probes and a microarray. A sample from a tumor of a person being analyzed is taken and the probes are added to the sample. Probes bind to the HER2 gene present in the sample. Bound probes are amplified and placed on the digital microarray, resulting in the amplicons binding to unique locations on the digital microarray. The microarray is then analyzed using processing circuitry and scanning circuitry. Each unique location of the microarray that is indicative of the HER2 gene is counted for the presence or absence of a fluorescent signal giving a binary result for each of the unique locations. The total number of the presence fluorescent signals is summed to provide a target count score for the HER2 gene. In some instances, a target count score is also provided for a total number of normal or other cells present. The target count score for the HER2 gene is indicative of the concentration of the HER2 gene in the sample and used to provide prognosis information and/or treatment information. For example, the copy number status of HER2 gene in a tumor can be useful to the effectiveness of treatment approaches as a number of drugs (e.g., Herceptin, Perjeta and Tykerb) are used to treat tumors that have an overexpression of the HER2 gene.

In some specific embodiments, the digital output is implemented using one or more apparatuses. The apparatus includes processing circuitry and scanning circuitry. The scanning circuitry is used to capture fluorescent signal intensities indicative of tag sequences bound to the microarray. The processing circuitry uses the captured fluorescent signal intensities to provide the digital output. In various embodiments, the apparatus additionally includes a microfluidic card with a plurality of chambers that are in fluidic connection and that are used to perform the hybridization of the probes to the targets in the sample, purification, and amplification (and optionally the hybridization of the amplicons to the microarray), such as the rapid assay apparatus illustrated by FIGS. 8A-8C, as well on page 2 of the underlying Provisional Application entitled “Rapid Assay Process Development” and on page 2 of the attached appendix of the underlying Provisional Application entitled “Digital Microassay”. In such an apparatus/microfluidic card, relevant chambers and/or modules are in fluidic communication so as to pass the sample from one chamber/module to the next for operating on the sample according to the functionality relevant thereto, such as the hybridization to probes, target purification, and amplification. In other embodiments, one or more additional apparatuses as used to perform the hybridization and amplification processes, such as various thermal cyclers. For example, the sample can be in fluidic movement through a plurality of chambers of a microfluidic card.

Example scanning circuitry includes a light source that emits a light beam (e.g., a polarizing light beam), an optical assembly, and detector circuitry. The optical assembly is configured to selectively optically interrogate the substrate, such as the above-described digital microarray (e.g., provide the beam of light to particular locations of the digital microarray). For example, the optical assembly has a surface adapted to allow placing thereon a substrate (e.g., a microarray). In other embodiments, the optical assembly includes digital micromirror device (DMP). Further, in specific embodiments, the optical assembly includes a mechanical mechanism, such as a wheel that the digital microarray is placed on that rotate and/or that rotates the location of the light beam on the digital light beam.

The light beam is selectively directed to particular locations of the substrate (e.g., digital microarray). For example, the light beam from the light source is reflected by the surface to provide an evanescent field over a location of the substrate (e.g., a digital microarray) such that the location of the digital microarray in the evanescent field causes a polarization change in the light beam. The scanning circuitry can include a confocal laser as the light beam.

The detection circuitry detects an optical signal in response to the light beam being selectively directed to locations of the substrate (e.g., a digital microarray). In specific embodiment, the detector circuitry is position to detect the polarization change in the light beam as the light beam is scanned over the substrate (e.g., a microarray). The polarization change in the light beam and/or the detected signal is indicative of the fluorescent signal at the particular location of the substrate. Processing circuitry is coupled to the detection circuitry to process an optical signal from the detection circuitry to obtain a representation of the fluorescent signal at the location of the substrate (e.g., the intensity of the fluorescent signal). Further, the processing circuitry processes a plurality of optical signals to obtain representations of florescent signals at a plurality of locations of the substrate. The detector circuitry can include various lens, optical wavelength guides. The scanning circuitry, in some instances, is and/or includes imaging circuitry, such as a charged coupled device (CCD).

In various embodiments, the processing circuitry is configured to perform repetitive comparative measurements of the optical signals from plurality of location of the substrate (e.g., a digital microarray). The processing circuitry uses the captured optical signals to provide the digital output, as previously described herein. Example scanner systems include the Tecan™ Power Scanner or the GenePix™ 4000B Microarray Scanner (e.g., a microarray scanner) and the processing circuitry can utilize various computer-readable medium to analyze the results of the microarray, such as the Array-Pro™ Analyzer or the GenePix™ Pro Microarray Analysis Software (e.g., Acuity™).

FIG. 7 illustrates an example apparatus used for assessing target sequence numbers, in accordance with various embodiments of the present disclosure. As described above, the apparatus includes processing circuitry 781 and scanning circuitry 782. The scanning circuitry 782 is used to capture (fluorescent) signal intensities indicative of tag sequences bound to the substrate 783, such as an above-described microarray. The processing circuitry 781 uses the captured signal intensities to provide the digital outputs.

As previously described, the substrate 783 has a plurality of complementary tag sequences at a plurality of different locations on a substrate (e.g., a microarray), which can be referred to as complementary tag locations. The complementary tag sequences are configured to bind to different probes. The sample is exposed to the plurality of probes, as previously described. For example, a plurality of sets of different probes can be placed in contact with a biological sample 784 from an organism. Example biological samples include blood, tissue, saliva, urine, etc., taken from an organism, such as a human. The probes in a set of probes for a particular target has a complementary target sequence configured to bind to a particular target in the sample 784, and a different (e.g., unique) tag sequences configured to bind to a particular locations of the plurality of locations on the substrate 783. The total number of probes placed in contact with the sample 784 can include a plurality of sets of probes. Each set of probes is designed for a different target sequence and used to assess a relative number of copies of the respective target sequence present in the biological sample 784.

The scanning circuitry 782 scans the substrate 783, and therefrom, captures the signals (e.g., optical intensities) indicative of a tag sequence bound to the substrate 783. The scanning circuitry 782 can provide the captured signals to the processing circuitry 781. The processing circuitry 781 uses the captured signals, in addition to information indicative of the different locations and associated tag sequences, to assess a number of each of the target sequences present in the sample 784, as previously described.

In specific embodiments, the apparatus illustrated by FIG. 7 is used to assess a plurality of different target sequences at the same time, such as 10 to 100,000 target sequences. In such embodiments, the substrate 783 has between 10 to 100,000 sets of complementary tag sequences, with each set being associated with one of the target sequences. In other embodiments, the substrate 783 has between 100 and 10,000 sets. The processing circuitry 781 can determine a concentration (e.g., copy number) of each of the plurality of target sequences by counting the number of each target sequence present on the substrate 783. For example, at each of the plurality of different locations on the substrate 783 known to be associated with a target sequence (e.g., has a complementary tag sequence configured to bind to a tag sequence of the set of probes for the respective target), the processing circuitry 781 determines if a copy of the target sequence is present at each of a plurality of complementary tag locations of substrate using the signals (e.g., fluorescent signal intensities) captured by the scanning circuitry 782, and which can be performed for each of the plurality of target sequences. The plurality of different complimentary tag locations are among the plurality of different locations and are associated with the respective target sequence (e.g., have a complement to a tag sequence of the set of probes for the target). The processing circuitry 781 sums the number of copies of the respective target sequence present on the substrate by increasing a target score count by one in response to determining a copy of the respective target sequence is present (e.g., “yes”) at one or more of the different complementary tag locations. In response to the particular complementary tag location having a signal intensity indicative of a target sequence not being present (e.g., a “no”), the target score count is not increased. The resulting target count scores (for each of the targets) can be compared to thresholds and used to diagnosis the organism that the sample is obtained from. As a non-limiting example, each threshold can be indicative of expected results for an organism that does not (or does) have a disease or other physiological disorder associated with the target sequence.

FIGS. 8A-8C illustrates another example apparatus used for assessing target sequence numbers, in accordance with various embodiments of the present disclosure. As previously described, the apparatus can include a microfluidic card with a plurality of chambers that are in fluidic connection and that are used to perform the hybridization of the probes to the targets in the sample, purification, and amplification (and optionally the hybridization of the amplicons to a substrate, such as a microarray), such as the rapid assay apparatus illustrated by FIGS. 8A-8C. In such an apparatus/microfluidic card, relevant chambers and/or modules are in fluidic communication so as to pass the sample from one chamber/module to the next for operating on the sample according to the functionality relevant thereto, such as the hybridization to probes, target purification, and amplification. In other embodiments, one or more additional apparatuses can be used to perform the hybridization and amplification processes, such as various thermal cyclers. For example, the sample can be in fluidic movement through a plurality of chambers of a microfluidic card.

It may also be helpful to appreciate the context/meaning of the following terms: sample refers to or includes a medium that contains one or more genomic targets to be analyzed; target refers to or includes a nucleic acid sequence to be analyzed; the terms “target”, “targets”, “target sequence”, or “genomic sequence” are used interchangeably throughout the disclosure; the terms “complementary sequence” and “complementary target sequence” can be used interchangeably throughout the disclosure; a probe or Molecular Inversion Probe refers to or includes a sequence used to analyze a target (e.g., the term “probe” is also used to mean the same as molecular inversion probe); the acronym MIP is used to indicate the same; tag refers to or includes a nucleic acid sequence within the larger sequence of the MIP that uniquely identifies that MIP molecule; the terms “binary result”, “digital result”, and “digital output” are used interchangeably throughout the disclosure; the term “complimentary tag sequence location” refers to or includes different locations on the substrate having a complimentary tag sequence located thereon (e.g., the term “different locations” or “unique locations” of the substrate can also be used to be the same as complimentary tag sequence locations; a set or a plurality of complimentary tag sequence location refers to or includes the set of different locations on the substrate having a complimentary tag sequence to a tag sequence associated with particular target sequence; the terms “different” location, probe, tag, tag sequence, target, complementary tag sequence, etc., refers to or includes a location, tag, and/or sequence that is different from a respective other location, tag, and/or sequence, and in specific examples can include unique or discrete locations, tags, and/or sequences (e.g., locations, tags, and/or sequences that are distinct from each of the other locations, tags, and/or sequences); and a substrate refers to or includes a surface or material having a plurality of genomic spots thereon. In specific embodiments, the substrate includes a glass, plastic and/or silicon substrate having a plurality of complementary tag sequences at different locations of and/or on a surface of the substrate. In other specific embodiments and/or in addition, the substrate includes an immuno-sandwich, a DNA chip and/or a biochip, such as multiple wells formed in an array on the substrate (e.g., a nanowell array or a microwell array).

Various embodiments are implemented in accordance with the underlying Provisional Application (Ser. No. 62/313,454), entitled “Rapid Assay Process Development”, filed Mar. 25, 2016, and underlying Provisional Application (Ser. No. 62/345,586), entitled “Digital Microassay”, filed on Jun. 3, 2016, to which benefit is claimed and are both fully incorporated herein by reference. For instance, embodiments herein and/or in the provisional application (including the appendices therein) may be combined in varying degrees (including wholly). For information regarding details of these and other embodiments, applications and experiments (as combinable in varying degrees with the teachings herein), reference may be made to the teachings and underlying references provided in the Provisional Applications and the attached Appendix which forms part of this patent document and is fully incorporated herein. Accordingly, the present disclosure is related to methods, applications and devices in and stemming from the disclosures in the attached Appendix (including the references and illustrations therein), and also to the uses and development of devices and processes discussed in connection with the references cited herein.

Certain embodiments are directed to a computer program product (e.g., nonvolatile memory device), which includes a machine or computer-readable medium having stored thereon instructions which may be executed by a computer (or other electronic device, such as processing circuitry or the scanning circuitry) to perform these operations/activities.

Various embodiments described above may be implemented together and/or in other manners. One or more of the items depicted in the present disclosure can also be implemented separately or in a more integrated manner, or removed and/or rendered as inoperable in certain cases, as is useful in accordance with particular applications. In view of the description herein, those skilled in the art will recognize that many changes may be made thereto without departing from the spirit and scope of the present disclosure.

Based upon the above discussion and illustrations, those skilled in the art will readily recognize that various modifications and changes may be made to the various embodiments without strictly following the exemplary embodiments and applications illustrated and described herein. As an example, the processing circuitry and the scanning circuitry can be part of separate devices and in communication via a wireless or wired link or can be part of the same device. Such modifications do not depart from the true spirit and scope of various aspects of the invention, including aspects set forth in the claims.

What is claimed is:

1. A method comprising:
exposing a sample to a plurality of probes, the plurality of probes including:
a plurality of complimentary sequences configured and arranged to bind to a plurality of target sequences in the sample, and
a plurality of different tag sequences for each of the plurality of probes directed to one of the plurality of target sequences in the sample, the different tag sequences being configured and arranged to bind to different locations on a substrate;
causing at least a portion of the plurality of target sequences bound to the probes to bind to the different locations on the substrate; and
by using scanning circuitry and information indicative of the different locations and associated tag sequences, assessing a number of the plurality of target sequences in the sample.

2. The method of claim 1, further including causing the plurality of probes to bind to at least a portion of the plurality of target sequences, the plurality of probes including a set of probes for each of the plurality of target sequences, wherein a set of probes for one of the plurality of target sequences includes M different tag sequences, and the substrate includes M different complementary tag sequences for each of the M different tag sequences.

3. The method of claim 1, further including assessing the number of the plurality of target sequences in the sample at a plateau stage of a reaction process associated with the target sequences bound to the probes.

4. The method of claim 1, further including determining a concentration of at least one of the plurality of target sequences in the sample based on a count of the number of the plurality of different tag sequences bound to the substrate using processing circuitry.

5. The method of claim 1, wherein assessing the number of the plurality of target sequences in the sample further includes capturing fluorescent signal intensities indicative of tag sequences bound to the substrate using the scanning circuitry and counting the number of the plurality of different tag sequences bound to the substrate based on the captured fluorescent signal intensities.

6. The method of claim 1, further including determining copy number variation using a count of the number of the plurality of different tag sequences bound to the substrate, the substrate further having complementary tag sequences on the substrate configured and arranged to bind to the respective tag sequences.

7. The method of claim 1, the substrate further including, at each of the different locations of the substrate, a complementary tag sequence configured to bind with a tag sequence directed to one of the plurality of target sequences, and the method further including:
determining if a copy of a respective one of the plurality of the target sequences is present at each of the plurality of different locations; and
increasing a target count score indicative of a copy number of the respective target sequence by one responsive to determining the copy of the respective target sequence is present at one complementary tag location of the plurality of different locations and not increasing the target count score in response to determining the copy of the target sequence is not present at the one complementary tag location.

8. The method of claim 1, further including summing the number of the different tag sequences present on the substrate to quantify target concentrations as a target count score and comparing the target count score for each of the target sequences to a threshold that is indicative of at least one of a diseased state, a prognosis, a diagnosis, a treatment, and a combination thereof.

9. The method of claim 1, wherein at least some of plurality of target sequences exhibit different sequences and the plurality of probes includes a set of probes for each of the different sequences of the plurality of target sequences and further including assessing the number of each of the different sequences of the plurality of target sequences in the sample at a plateau stage of a reaction process associated with the different sequences.

10. An apparatus comprising:
a substrate having a plurality of complementary tag sequences at a plurality of different locations on the substrate, the complementary tag sequences being configured and arranged to bind to a plurality of tag sequences as part of a plurality of probes;
the plurality of probes including a set of probes for each of a plurality of target sequences, the set of probes for one of the plurality of target sequences including:
a plurality of copies of a complimentary sequence configured and arranged to bind to the target sequence, and
a plurality of different tag sequences configured and arranged to bind to a particular location of the plurality of different locations on the substrate;
scanning circuitry configured and arranged to scan the substrate and, therefrom, capture signals indicative of tag sequences bound to the substrate; and
processing circuitry configured and arranged to assess a number of each of the plurality of target sequences in a sample based on the captured signals and information indicative of the plurality of different locations and associated with the plurality of tag sequences.

11. The apparatus of claim 10, wherein:
the scanning circuitry is configured and arranged to capture fluorescent signal intensities indicative of tag sequences bound to the substrate; and
the processing circuitry configured and arranged to provide a digital output for each of the plurality of different locations using the captured fluorescent signal intensities and the information indicative of the plurality of different locations and the respective tag sequences of the plurality of different tag sequences.

12. The apparatus of claim 10, wherein the substrate is a microarray having a set of complementary tag sequences bound at the plurality of different locations for each of the plurality of tag sequences.

13. The apparatus of claim 12, wherein the microarray is configured and arranged with at least 10 sets of complementary tag sequences used to assess at least 10 different target sequences.

14. The apparatus of claim 13, wherein the processing circuitry is configured and arranged to assess the number of the at least 10 different target sequences at a plateau stage of a polymerase chain reaction (PCR) reaction associated therewith.

15. The apparatus of claim 10, wherein the processing circuitry is configured and arranged to determine a concentration of each of the plurality of target sequences in the sample based on a count of the number of each of the plurality of tag sequences present on the substrate.

16. The apparatus of claim 10, wherein the processing circuitry is configured and arranged to determine a target count score for each of the plurality of target sequences by:
determining if a copy of a respective target sequence is present at each of a plurality of different complimentary tag locations on the substrate and using fluorescent signal intensities captured by the scanning circuitry, the plurality of different complimentary tag locations being among the plurality of different locations and associated with the respective target sequence, and
summing the number of copies present on the substrate by increasing a target count score by one responsive to determining the copy of the respective target sequence is present at one or more of the plurality of different complimentary tag locations.

17. The apparatus of claim 16, wherein the processing circuitry is configured and arranged to compare the target count scores to thresholds, each threshold being indicative of expected results for an organism that does not have a disease or other physiological disorder associated with one or more of the plurality of target sequences.

18. The apparatus of claim 10, wherein the plurality of complementary tag sequences are arranged at the plurality of different locations on the substrate, each location being unique to one of the plurality of probes configured and arranged to bind to the respective complementary tag sequences.
